(19) United States
(12) Patent Application Publication
(10) Pub. No.: US 2015/0105675 A1
(43) Pub. Date: Apr. 16, 2015

(54) SPHYGMOMANOMETER

(71) Applicant: DENSO CORPORATION, Kariya-city (JP)

(72) Inventors: Tsuyoshi NAKAGAWA, Aichi-gun (JP); Taiji KAWACHI, Kariya-city (JP); Kouki FUTATSUYAMA, Anjo-city (JP); Hiroshi YAMAKITA, Ichinomiya-city (JP)

(21) Appl. No.: 14/510,329

(22) Filed: Oct. 9, 2014

(30) Foreign Application Priority Data
Oct. 16, 2013 (JP) 2013-215648

(51) Int. Cl. A61B 5/021; A61B 5/00; A61B 5/022

(52) U.S. Cl. CPC A61B 5/02125; A61B 5/022; A61B 5/7203
USPC 600/493

(57) ABSTRACT

A sphygmomanometer includes an operation part, a pulse wave sensor that detects a pulse wave signal indicative of a pulse wave in a part of the patient body that is operating the operation part. A first manometer measures a blood pressure based on the pulse wave signal, and a second manometer measures the blood pressure with a cuff that is worn by the patient. A cuff wearing detector detects a wearing of the cuff by the patient. A controller measures the blood pressure with the first manometer when the operation part is operated long enough for a detection of the pulse wave signal and measures the blood pressure with the second manometer when the cuff wearing detector detects that the cuff is worn and that the operation part is being operated.

### CROSS REFERENCE TO RELATED APPLICATION

The present application is based on and claims the benefit of priority of Japanese Patent Application No. 2013-215648, filed on Oct. 16, 2013, the disclosure of which is incorporated herein by reference.

### TECHNICAL FIELD

The present disclosure generally relates to a sphygmomanometer.

### BACKGROUND INFORMATION

Conventionally, a sphygmomanometer is a known measurement method for measuring blood pressure based on a pulse wave signal of a patient that is detected by a pulsewave sensor (hereafter referred to as a “cuff-less measurement”).

This sphygmomanometer also has a function which measures a blood pressure by using a cuff (hereafter referred to as a “cuff measurement”). The cuff-less measurement and the cuff measurement may be performed simultaneously on the same patient, and the difference between the measured values in both measurement methods may be computed. Then, by using the computed difference, the measured value of the cuff-less measurement performed thereafter may be corrected according to a patent document 1 (i.e., Japanese Patent No. 3422128).

The sphygmomanometer is provided with two separate operation switches, that is, an operation switch for performing the cuff-less measurement and an operation switch for performing the cuff measurement, respectively. Therefore, in order to perform the cuff-less measurement and the cuff measurement simultaneously, two operation switches are required to be operated.

### SUMMARY

It is an object of the present disclosure to provide a sphygmomanometer that is easily operable.

In an aspect of the present disclosure, the sphygmomanometer includes an operation part that is operated by a part of a patient body, a pulse wave sensor detecting a pulse wave signal indicative of a pulse wave in the part of the patient body that is operating the operation part, a first manometer measuring a blood pressure based on the pulse wave signal detected by the pulse wave sensor, a second manometer measuring the blood pressure with a cuff that is worn by the patient, and a cuff wearing detector detecting a wearing of the cuff by the patient. The sphygmomanometer also includes a controller measuring the blood pressure with the first manometer when the operation part is being operated and a duration of the detection of the pulse wave signal by the pulse wave sensor is equal to or greater than a preset time, and measuring the blood pressure with the second manometer when the operation part is being operated and the cuff wearing detector detects the wearing of the cuff.

Further, according to the sphygmomanometer of the present disclosure, a difference calculator calculating and memorizing a difference between a measurement value of the first manometer and a measurement value of the second manometer, and a correction part correcting the measurement value according to the difference.

Additionally, according to the sphygmomanometer of the present disclosure, the operation part is operated by a first step and a second step, and a direction to operate the first step and a direction to operate the second step is a same direction, when the first step is operated, the controller performs a measurement by using one or both of the first manometer and the second manometer, and when the second step is operated, the difference calculator performs a calculation and memorizes the difference.

Even further, according to the sphygmomanometer of the present disclosure, the cuff wearing detector detects the wearing of the cuff based on a circumferential stretch amount of the cuff.

Moreover, according to the sphygmomanometer of the present disclosure, the part of the patient body is a part of an arm that is opposite to a cuff-wearing arm.

In another aspect of the present disclosure, the sphygmomanometer includes an operation part that is operated by a part of a patient body, a pulse wave sensor detecting a pulse wave signal indicative of a pulse wave in the part of the patient body that is operating the operation part, a first manometer measuring a blood pressure based on the pulse wave signal detected by the pulse wave sensor, a second manometer measuring the blood pressure with a cuff that is worn by the patient, and a cuff wearing detector detecting a wearing of the cuff by the patient. The sphygmomanometer also includes a controller measuring the blood pressure with the first manometer when the operation part is being operated and a duration of the detection of the pulse wave signal by the pulse wave sensor is equal to or greater than a preset time, measuring the blood pressure with the second manometer when the operation part is being operated, a duration of the detection of the pulse wave signal by the pulse wave sensor is shorter than the preset time, and the cuff wearing detector detects the wearing of the cuff, or measuring the blood pressure with the first manometer and the second manometer when the operation part is being operated, a duration of the detection of the pulse wave signal by the pulse wave sensor is equal to or greater than a preset time, and the cuff wearing detector detects the wearing of the cuff.

According to the sphygmomanometer of the present disclosure, a controller in the sphygmomanometer controls the first and second manometers in the following manner. That is, when the operation of the operation part is determined as a longer-than-preset-time detection of the pulse wave signal with the pulse wave sensor, the blood pressure is measured by the first manometer, and when (a) the cuff wearing detector detects a wearing of the cuff and (b) the operation part is being operated, the blood pressure is measured by the second manometer.

According to the sphygmomanometer of the present disclosure, based on an operation of the operation part performed by a patient, the first manometer is used to measure the blood pressure (i.e., the cuff-less measurement), and, simultaneously, the second manometer is used to measure the blood pressure (i.e., the cuff measurement), which provides an ease of use for a patient using the sphygmomanometer.

Further, according to the sphygmomanometer of the present disclosure, when the duration of the detection of the pulse wave signal by the pulse wave sensor is controlled to be shorter than the preset time (e.g., when the operation part is operated only for a short time), the cuff-less measurement will not be performed. Further, according to the sphygmomanometer of the present disclosure, when the cuff is not worn, the cuff measurement will not be performed.

### BRIEF DESCRIPTION OF THE DRAWINGS

Objects, features, and advantages of the present disclosure will become more apparent from the following detailed description made with reference to the accompanying drawings, in which:

FIG. 1 is a block diagram of a configuration of a sphygmomanometer;

FIG. 2A is a perspective view of the configuration of the sphygmomanometer in a non-wearing state, in which a cuff of the sphygmomanometer is not worn by a patient;

FIG. 2B is a perspective view of the configuration of a wear state of the cuff which is worn by the patient;

FIG. 3 is a plan view of a configuration of a measurement switch;

FIG. 4 is a side section view of a configuration of the measurement switch;

FIG. 5 is a side section view of a configuration of a pulse wave sensor;

FIG. 6 is a flowchart of a main process performed by the sphygmomanometer;

FIG. 7 is a flowchart of a cuff/cuff-less measurement process performed by the sphygmomanometer;

FIG. 8 is a flowchart of a cuff-less measurement process performed by the sphygmomanometer;

FIG. 9 is a side section view of the configuration of the measurement switch in a second embodiment;

FIG. 10A is a perspective view of the configuration of the sphygmomanometer in a non-wearing state, in which the cuff of the sphygmomanometer is not worn by the patient; and

FIG. 10B is a perspective view of the configuration of a wear state of the cuff which is worn by the patient.

### DETAILED DESCRIPTION

Embodiments of the present disclosure are described based on the drawings.

### First Embodiment

1. Configuration of Sphygmomanometer 1

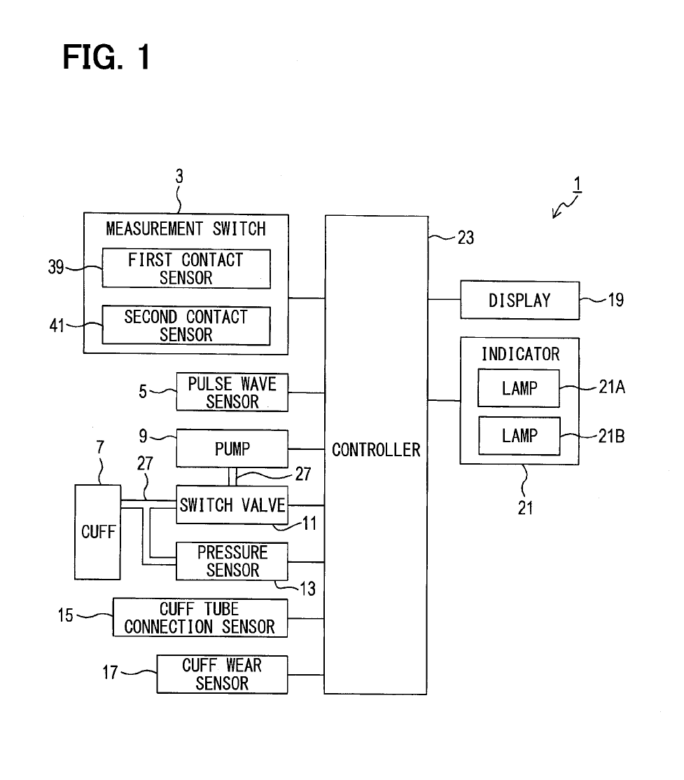

The configuration of the sphygmomanometer 1 is described based on FIGS. 1-5. The sphygmomanometer 1 is provided with a measurement switch 3, a pulse wave sensor 5, a cuff 7, a pump 9, a switch valve 11, a pressure sensor 13, a cuff tube connection sensor 15, a cuff wear sensor 17, a display 19, an indicator 21, and a controller 23 as shown in FIG. 1.

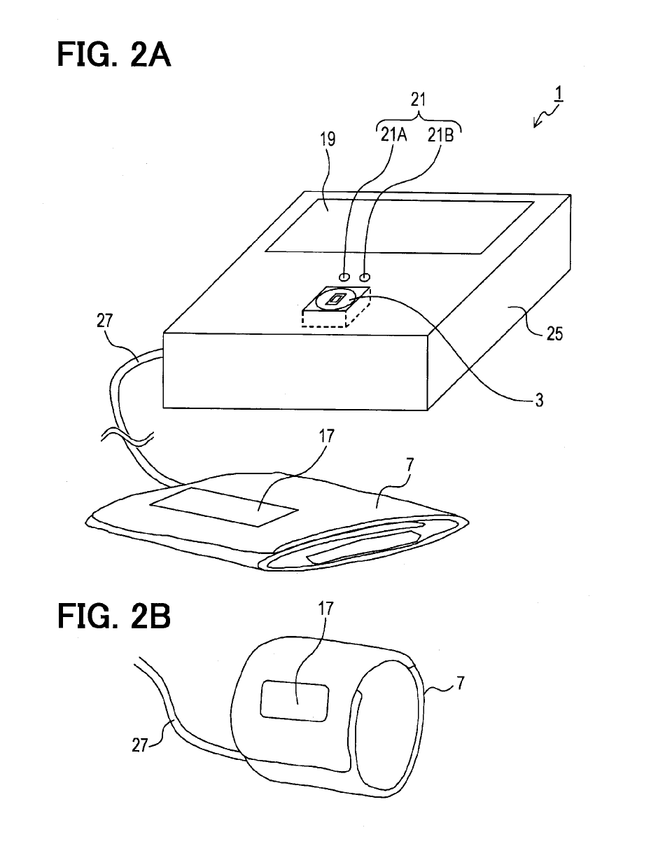

Further, the sphygmomanometer 1 is provided having a box shape case 25 as shown in FIG. 2, and the measurement switch 3, the display 19, and the indicator 21 are arranged on a surface of the case 25. A tube 27 which supplies air to the cuff 7 via the switch valve 11 from the pump 9 extends from the case 25. Further, the cuff wear sensor 17 is attached onto an outer circumference side of the cuff 7.

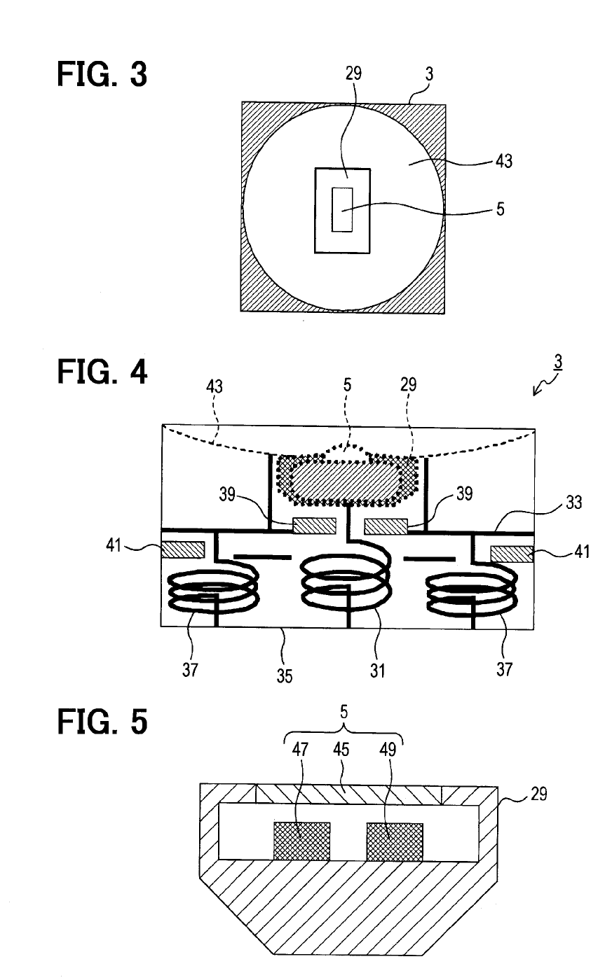

The measurement switch 3 is a press switch which is operated by a patient in a press-down manner, i.e., the patient operates the switch 3 with his/her finger. The measurement switch 3 is provided with an operation piece 29, a spring 31, a stopper 33, a pedestal section 35, a spring 37, a first contact sensor 39, a second contact sensor 41, and a switch housing 43 as shown in FIG. 3 and FIG. 4.

The operation piece 29 is attached to the pedestal section 35 via the spring 31, and is operable, i.e., is pressed by a patient finger to move in a downward direction (i.e., downward in FIG. 2 and FIG. 4) that allows an approach and contact of the operation piece 29 with the pedestal section 35. When the patient finger is lifted from a pressing state of pressing the operation piece 29, the operation piece 29 returns to an original position according to the elastic force of the spring 31.

A stopper 33 is a tabular member disposed at a position between the pedestal section 35 and the operation piece 29. On the stopper 33, the first contact sensor 39 is disposed at a position that faces the operation piece 29. The first contact sensor 39 is away and separate from the operation piece 29 when the operation piece 29 is not pressed, and, when the operation piece 29 is pressed by a preset amount or more, the first contact sensor 39 contacts the operation piece 29 for outputting a signal that indicates a contact therebetween (i.e., designated as a first contact signal hereafter).

Further, the stopper 33 is disposed on the pedestal section 35 via the spring 37, and is operable, i.e., is pressed to move toward the pedestal section 35 (i.e., moves in the same direction as the operation piece 29 when being pressed). When pressing of the stopper 33 is stopped in a situation in which the stopper 33 is being pressed, the stopper 33 returns to an original position according to the elastic force of the spring 37.

The second contact sensor 41 is disposed at a position underneath the stopper 33. The second contact sensor 41 is away and separate from the stopper 33 when the stopper 33 is not in a pressed state, and, when the stopper 33 is pressed by a preset amount or more, the second contact sensor 41 contacts the stopper 33 and outputs a signal (i.e., designated hereafter as a second contact signal).

The switch housing 43 contains each of the components of the measurement switch 3 in an inside thereof. However, a part of a center section in an upper surface of the switch housing 43 is cut out, and the operation piece 29 is exposed therefrom. The upper surface of the switch housing 43 is configured to have a concave shape, i.e., a bowl shape, and the operation piece 29 is located at the center and the bottom of such concave shape.

The pulse wave sensor 5 is disposed on a surface side (i.e., a surface for a contact with the patient finger) of the operation piece 29. The patient finger operating the operation piece 29 thus contacts the pulse wave sensor 5. That is, the pulse wave sensor 5 is disposed at a position which enables a detection of a pulse wave signal from the patient finger which operates the measurement switch 3.

The pulse wave sensor 5 is provided with a lens 45, a light-emitting part 47, and a light sensing part 49 as shown in FIG. 5. The lens 45 is disposed to serve as an upper-most surface of the operation piece 29, through which a light passes in an up-down direction. The light-emitting part 47 and the light sensing part 49 are located under the lens 45 (i.e., in an inside of the operation piece 29). The light-emitting part 47 comprises a light emitting diode, and irradiates a green light (i.e., a light in the wavelength of 5000 A-8000 A [Angstroms]) in an upward direction through the lens 45. The irradiated light is then reflected in a capillary vessel of the patient finger positioned on the lens 45. The reflected light passes through the lens 45, and is received by the light sensing part 49. The light sensing part 49 is a photo diode, and, when the above-mentioned light is received, it outputs an electric signal (i.e., a pulse wave signal).

The measurement switch 3 having the above-described configuration operates in the following manner according to an operation performed by the patient. That is, in the measurement switch 3 in a non-operation state, i.e., when the operation piece 29 is not being pressed by the patient finger, both of the first contact sensor 39 and the second contact sensor 41 do not output a contact signal.

When the operation piece 29 is gradually pressed down by the patient finger, soon, the operation piece 29 will contact the first contact sensor 39, and then the first contact sensor 39 outputs the first contact signal. At such time of contact of the operation piece 29 with the first contact sensor 39, the second contact sensor 41 does not yet output the second contact signal. This step of operation to operate the measurement switch 3 is designated as a first step operation hereafter.

When the operation piece 29 is further pressed down in the same direction, the stopper 33 being depressed by the operation piece 29 contacts the second contact sensor 41, and the second contact sensor 41 outputs the second contact signal. At such time of contact of the stopper 33 with the second contact sensor 41, the first contact sensor 39 is also outputting the first contact signal. This step of operation to operate the measurement switch 3 is designated as a second step operation.

When an amount of pressing of the operation piece 29 is decreased from a second step operation state, the second step operation state is changed back to (i.e., returns to) a first step operation state, and returns further to the non-operation state.

The cuff 7 is structured as a rubber bag contained in a cloth-made band-shape outer bag, which may be wrapped around an upper arm of the patient. The pump 9 supplies air into an inside of the cuff 7 through the tube 27. The switch valve 11 may be switchable among three states, i.e., (i) a state of allowing a supply of air into the cuff 7, (ii) a state of gradual release of air from the cuff 7, and (iii) a state of very quick release of air from an inside of the cuff 7. The pressure sensor 13 detects the pressure in the cuff 7.

The cuff 7 may preferably be wrapped around an opposite arm that is opposite to an arm of the patient which operates the operation piece 29. For example, when a patient operates the operation piece 29 with a finger of the right hand, the cuff 7 may preferably be wrapped around the left arm.

The cuff tube connection sensor 15 is a sensor which detects a connection state between the tube 27 and a main part of the sphygmomanometer 1 (i.e., the sphygmomanometer 1 other than the cuff 7). That is, the sensor 15 detects/determines whether the tube 27 is connected to the sphygmomanometer 1.

The cuff wear sensor 17 is a sensor which detects whether the cuff 7 is wrapped around a patient's upper arm (i.e., for detecting a wear state). The cuff wear sensor 17 is made of a conductive rubber in a thin plate shape (i.e., a rubber mixed with a conductive material), and as mentioned above, disposed onto an outer circumference side of the cuff 7.

The cuff wear sensor 17 detects the wear state of the cuff 7 based on the following principles. That is, as shown in FIG. 2A, when the patient does not wear the cuff 7 on his/her upper arm, a pulling force does not act on the cuff wear sensor 17, and does not stretch the cuff wear sensor 17.

On the other hand, as shown in FIG. 2B, when the patient wears the cuff 7 on his/her upper arm, the pulling force acts on the cuff wear sensor 17, and the sensor 17 stretches along a circumferential direction of the cuff 7.

Further, since an electrical resistance of the conductive rubber drastically changes according to an amount of stretch of the rubber (i.e., according to an amount of displacement), the electrical resistance of the rubber in FIG. 2A and the electrical resistance of the rubber in FIG. 2B are different. Therefore, the cuff wear sensor 17 is capable of detecting the wear state of the cuff 7 (i.e., whether the patent wears the cuff 7 on his/her upper arm) based on the change of the electrical resistance (i.e., based on the change of the amount of stretch).

The display 19 is a well-known liquid crystal display, and is capable of displaying various images. The indicator 21 comprises two lamps 21A and 21B. The lamp 21A is a blue light lamp which emits a blue light, and the lamp 21B is a red light lamp which emits a red light. The indicator 21 indicates a state of the sphygmomanometer 1 by using various lighting patterns of the lamp 21A and the lamp 21B, i.e., based on a light state, an extinguished state, and/or a blink state of the lamps 21A/B. As for various states of the sphygmomanometer 1, the following states are at least indicated. That is, by using the lamps 21A/B, the display 19 shows a cuff-less measurement state, a cuff-less measurement NG state, a measurement complete state, a difference Δ memory calculate/memory complete state, a switch 3 non-operation state, and the like.

The controller 23 is a well-known computer having CPU, ROM, RAM, etc. The controller 23 performs various processes which control the components of the sphygmomanometer 1 to be mentioned later. Especially, the controller 23 detects a pulse wave signal from the patient finger by using the pulse wave sensor 5 during an output period of the first contact signal from the first contact sensor 39. That is, a time duration in which the first contact signal is being outputted and a time duration in which the pulse wave sensor 5 detects a pulse wave signal match with each other.

In the description of the above embodiment, the patient finger corresponds to a part of a patient body in the claims, and the measurement switch 3 corresponds to an operation part in the claims, and the controller 23 corresponds to a first manometer, a second manometer, a controller, a difference calculator, and a correction part in the claims. The cuff wear sensor 17 corresponds to a cuff wearing detector in the claims.

2. Process Performed by Sphygmomanometer 1

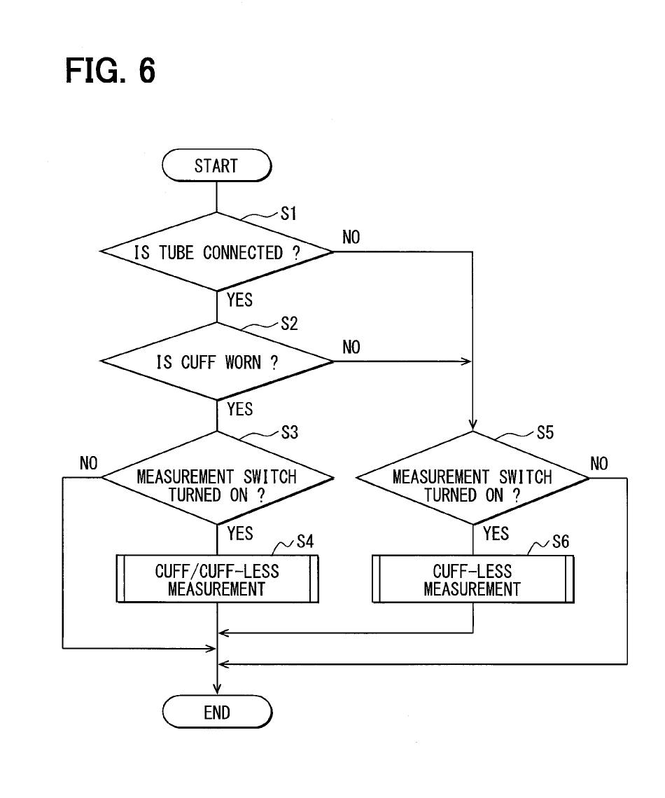

The process performed by the sphygmomanometer 1 (especially, by the controller 23) is described based on the flowchart of FIGS. 6-8. This process is repeated at predetermined intervals during a power ON time of the sphygmomanometer 1.

In Step 1 of FIG. 6, it is determined using the cuff tube connection sensor 15 whether the tube 27 is connected with the main part of the sphygmomanometer 1. The process proceeds to Step 2 when the tube 27 is connected, and the process proceeds to Step 5 when the tube 27 is not connected.

In Step 2, it is determined using the cuff wear sensor 17 whether the patient wears the cuff 7. When patient wears the cuff 7, the process proceeds to Step 3, and, when the patient does not wears the cuff 7, the process proceeds to Step 5.

In Step 3, it is determined whether the first step operation is performed on the measurement switch 3 (i.e., whether the measurement switch 3 is outputting the first contact signal or not). When the first step operation is performed, the process proceeds to Step 4, and, when the first step operation is not performed, this process is ended.

In Step 4, the cuff/cuff-less measurement is performed. The cuff/cuff-less measurement is mentioned in detail later.

In Step 5, it is determined whether the first step operation is performed on the measurement switch 3 (i.e., whether the measurement switch 3 is outputting the first contact signal or not). When the first step operation is performed, the process proceeds to Step 6, and, when the first step operation is not performed, this process is ended.

In Step 6, the cuff-less measurement is performed. The cuff-less measurement is mentioned in detail later.

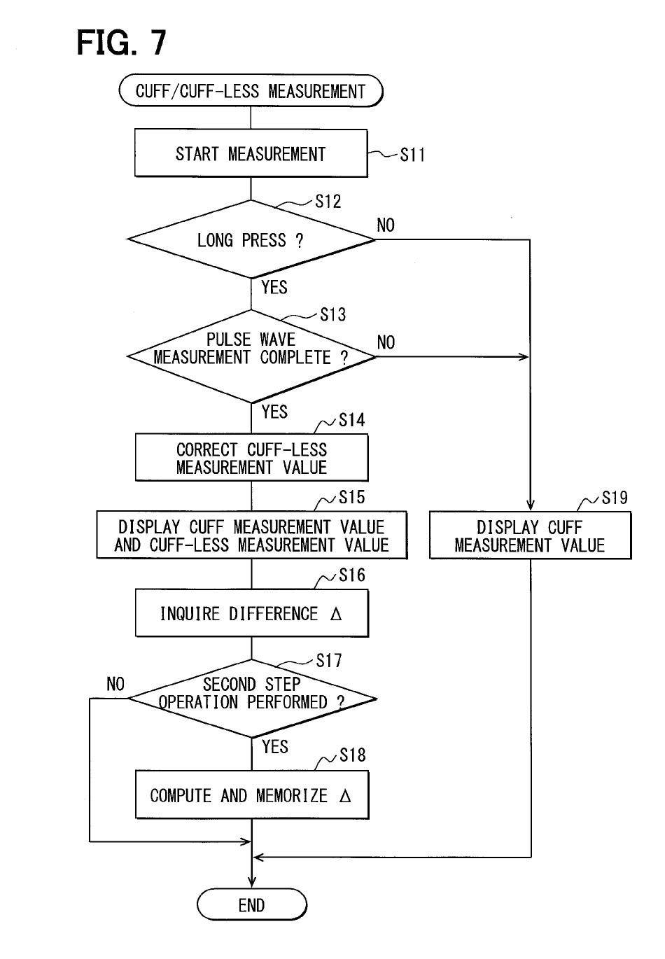

Next, based on the flowchart of FIG. 7, the cuff/cuff-less measurement in Step 4 is described in detail. In Step 11, the cuff measurement and the cuff-less measurement are started, respectively. The cuff measurement is a blood pressure measurement in a well-known method, using the cuff 7. For example, the cuff measurement may be performed in the following manner. When a compression pressure of the cuff 7 which is wrapped around the patient's upper arm is changed, a magnitude of the pulse wave during such change of the compression force is also changed, based on which the blood pressure of the patient is automatically measured. That is, after quickly raising the compression pressure of the cuff 7 (e.g., up to 180 mmHg), the compression pressure of the cuff 7 is gradually decreased by about 3 mmHg/sec, which may be designated as a gradual pressure decrease period, and during such a gradual pressure decrease period, an oscillometric method utilizing an amplitude change of the pulse wave that is represented by the pulse wave signal taken from the cuff 7 is used to determine the minimum and maximum blood pressures, for example.

The cuff-less measurement is a measurement of a blood pressure value based on the pulse wave signal of the patient which is detected by the pulse wave sensor 5. Specifically, a characteristic quantity is computed from the pulse wave signal, the characteristic quantity is applied to a blood-pressure inference model, and a measurement value of the blood pressure is determined. In such a case, the pulse wave signal is obtained from the finger of the patient which is operating the operation piece 29 (i.e., which abuts on the operation piece 29).

In Step 12, it is determined whether a first step operation time during which the first step operation of the measurement switch 3 is performed by the patient (i.e., a duration of time during which the pulse wave sensor 5 detects the patient's pulse wave signal in an operation piece operated state where the patient's finger is pressing the operation piece 29) is equal to or greater than a predetermined threshold. When the first step operation time is longer than the predetermined threshold (i.e., S12:YES=LONG PRESS), the process proceeds to Step 13, and, when the first step operation time is shorter than the predetermined threshold, the process proceeds to Step 19.

In Step 13, it is determined whether a pulse wave measurement is complete (i.e., whether a duration of detection of the pulse wave signal is long enough for the cuff-less measurement). When the pulse wave measurement is complete, the process proceeds to Step 14, and, when the pulse wave measurement is not complete, the process proceeds to Step 19.

In Step 14, a measurement value of the cuff-less measurement (i.e., designated hereafter as a cuff-less measurement value) started in Step 11 is computed first. Then, the cuff-less measurement value is corrected by adding a difference Δ to the measurement value of the cuff-less measurement value.

Here, the difference Δ is more specifically defined in the following manner. That is, when the cuff-less measurement and the cuff measurement are performed for the same patient substantially at the same time, designating (i) M1 as a cuff-less measurement value and (ii) M2 as a measurement value of the cuff measurement (i.e., designated hereafter as a cuff measurement value), the difference Δ is a value derived by subtracting the value M1 from the value M2. The difference Δ is computed at Step 18 mentioned later, and is memorized in advance by a not-illustrated memory of the controller 23.

In Step 15, the display 19 displays the cuff measurement value and the cuff-less measurement value. The cuff-less measurement value to be displayed is a corrected value after correction in Step 14. However, when no difference Δ is memorized in the memory, the cuff-less measurement value without correction is displayed.

In Step 16, an inquiry inquiring the patient about whether to compute the difference Δ is displayed on the display 19.

In Step 17, it is determined whether the second step operation is performed on the measurement switch 3 within a predetermined time from the display in Step 16.

When the second step operation is performed, the process proceeds to Step 18, and, when the second step operation is not performed, this process is ended.

In Step 18, the difference Δ is computed by subtracting the cuff-less measurement value M1 from the cuff measurement value M2 which is most recently measured, and memorizes the difference Δ in the not-illustrated memory of the controller 23. When a difference Δ is already memorized in the memory, a new value overwrites the old one. This difference Δ is used for the correction in Step 14 performed thereafter.

On the other hand, when a negative determination has been made in Step 12 or Step 13, the process proceeds to Step 19 and the cuff measurement value regarding the cuff measurement started in Step 11 is displayed on the display 19.

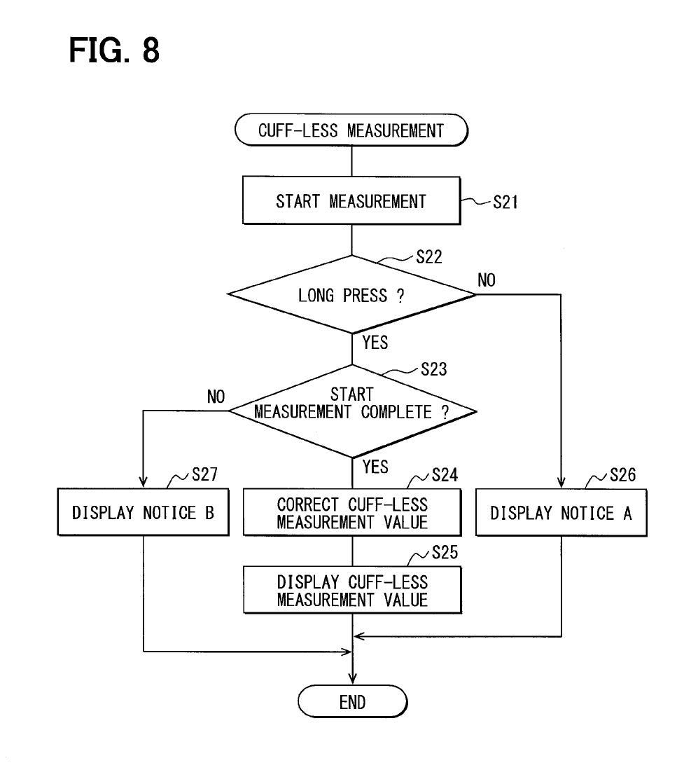

Next, based on the flowchart of FIG. 8, the cuff-less measurement of Step 6 is described in detail.

The cuff-less measurement is started in Step 21.

This cuff-less measurement is the same as the one that is performed in Step 11.

In Step 22, it is determined whether the first step operation time during which the first step operation of the measurement switch 3 is performed by the patient (i.e., a duration of time during which the pulse wave sensor 5 detects the patient's pulse wave signal in an operation piece operated state where the patient's finger is pressing the operation piece 29) is equal to or greater than a predetermined threshold. When the first step operation time is longer than the predetermined threshold (i.e., S22:YES=LONG PRESS), the process proceeds to Step 23, and, when the first step operation time is shorter than the predetermined threshold, the process proceeds to Step 26.

In Step 23, it is determined whether the pulse wave measurement is complete (i.e., whether a duration of detection of the pulse wave signal is long enough for the cuff-less measurement). When the pulse wave measurement is complete, the process proceeds to Step 24, and, when the pulse wave measurement is not complete, the process proceeds to Step 27.

In Step 24, a measurement value of the cuff-less measurement (i.e., the cuff-less measurement value) started in Step 21 is computed first. Then, the cuff-less measurement value is corrected by adding the difference Δ to the measurement value of the cuff-less measurement value.

In Step 25, the display 19 displays the cuff-less measurement value computed in Step 24. The cuff-less measurement value to be displayed is a corrected value after correction in Step 24. However, when no difference Δ is memorized in the memory, the cuff-less measurement value without correction is displayed.

On the other hand, when a negative determination is made in Step 22, the process proceeds to Step 26, and displays on the display 19 a notice A “For performing the cuff-less measurement, hold down (i.e., long-press) the button.”

When a negative determination is made in Step 23, the process proceeds to Step 27, and displays on the display 19 a notice B “Measurement time is not long enough.”

3. Effects of Sphygmomanometer 1

(1) According to the sphygmomanometer 1 of the present disclosure, the cuff-less measurement and the cuff measurement are performed (simultaneously) when the patient operates the measurement switch 3. Therefore, the operation of the sphygmomanometer 1 is easy for the patient.

(2) According to the sphygmomanometer 1 of the present disclosure, when the first step operation is performed on the measurement switch 3 only for a short time (i.e., the first step operation time in Step 12 shorter than the threshold, leading to the negative determination), the cuff measurement is performed while not performing the cuff-less measurement.

(3) According to the sphygmomanometer 1 of the present disclosure, when the measurement switch 3 is operated in a non-wearing state where the patient does not wear the cuff 7, the negative determination follows in Step 2, and the cuff-less measurement is performed while not performing the cuff measurement.

(4) The sphygmomanometer 1 of the present disclosure corrects the cuff-less measurement value by using the difference Δ. Therefore, a cuff-less measurement value having an improved accuracy is obtained.

(5) According to the sphygmomanometer 1 of the present disclosure, after performing one or both of the cuff measurement and the cuff-less measurement by performing the first step operation on the measurement switch 3, the operation piece 29 is further pressed down in the same direction (i.e., by performing the second step operation) for the computation and memory of the difference Δ.

(6) The sphygmomanometer 1 of the present disclosure has the cuff wear sensor 17, which enables the detection of the wear state of the cuff 7 on the patient by a simple detector configuration.

(7) As shown in FIG. 4, the upper surface of the switch housing 43 is configured to have a concave shape, i.e., a bowl shape, and the operation piece 29 is located at the center and the bottom of such concave shape. Therefore, an input of an external light which may interfere with the pulse wave detection into the pulse wave sensor 5 is prevented. Further, the above shape of the upper surface of the switch housing 43 as well as the position of the pulse wave sensor 5 naturally guide the patient finger to a right position (i.e., a position facing the sensor 5) for the pulse wave sensing.

(8) The patient can operate the operation piece 29 with the finger of an arm that is opposite to the arm wearing the cuff 7. Therefore, the influence of the cuff 7 on the pulse wave signal is minimized/reduced. Further, according to the blood pressure correctly estimated in such manner based on the pulse wave signal, an accuracy of computation of the difference Δ is further improved.

### Second Embodiment

1. Configuration of Sphygmomanometer 1

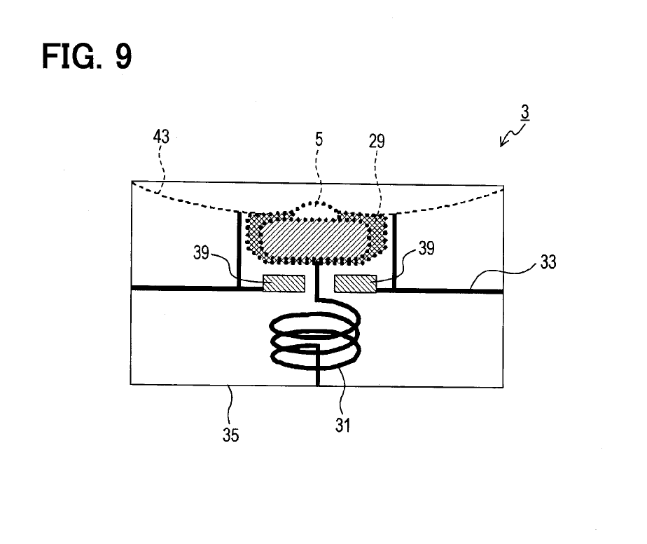

The configuration of the sphygmomanometer 1 in the second embodiment is the same as that of the first embodiment fundamentally. However, in the present embodiment, the configuration of the measurement switch 3 is shown in FIG. 9. The measurement switch 3 of the present embodiment is provided with the operation piece 29, the spring 31, the stopper 33, the pedestal section 35, the first contact sensor 39, and the switch housing 43.

The operation piece 29 is attached to the pedestal section 35 via the spring 31, and is operable, i.e., is pressed in by the patient finger to move in a downward direction that allows an approach and contact of the operation piece 29 with the pedestal section 35. When the patient finger is lifted from a pressing state of pressing the operation piece 29, the operation piece 29 returns to an original position according to the elastic force of the spring 31.

The stopper 33 is a tabular member disposed at a position between the pedestal section 35 and the operation piece 29. On the stopper 33, the first contact sensor 39 is disposed at a position that faces the operation piece 29. The first contact sensor 39 is away and separate from the operation piece 29 when the operation piece 29 is not pressed, and, when the operation piece 29 is pressed by a preset amount or more, the first contact sensor 39 contacts the operation piece 29 for outputting the first contact signal that indicates a contact therebetween. The stopper 33 is immovably fixed onto the switch housing 43.

The switch housing 43 contains each of the components of the measurement switch 3 in an inside thereof. However, a part of a center section in an upper surface of the switch housing 43 is cut out, and the operation piece 29 is exposed therefrom. The upper surface of the switch housing 43 is configured to have a concave shape, i.e., a bowl shape, and the operation piece 29 is located at the center and the bottom of such concave shape.

The pulse wave sensor 5 is disposed on a surface side (i.e., a surface for a contact with the patient finger) of the operation piece 29, which is the same sensor as the one in the first embodiment.

The measurement switch 3 having the above-described configuration operates in the following manner according to an operation performed by the patient. That is, in the measurement switch 3 in a non-operation state, i.e., when the operation piece 29 is not being pressed by the patient finger, the first contact sensor 39 does not output the first contact signal.

When the operation piece 29 is gradually pressed down by the patient finger, soon, the operation piece 29 will contact the first contact sensor 39, and then the first contact sensor 39 outputs the first contact signal. This step of operation to operate the measurement switch 3 is designated as the first step operation.

When an amount of pressing of the operation piece 29 is decreased from an amount of the first step operation, the first step operation is changed back to (i.e., returns to) to the non-operation state. In other words, the measurement switch 3 is operable to perform the first step operation. The measurement switch 3 in the present embodiment is not operable to perform the second step operation.

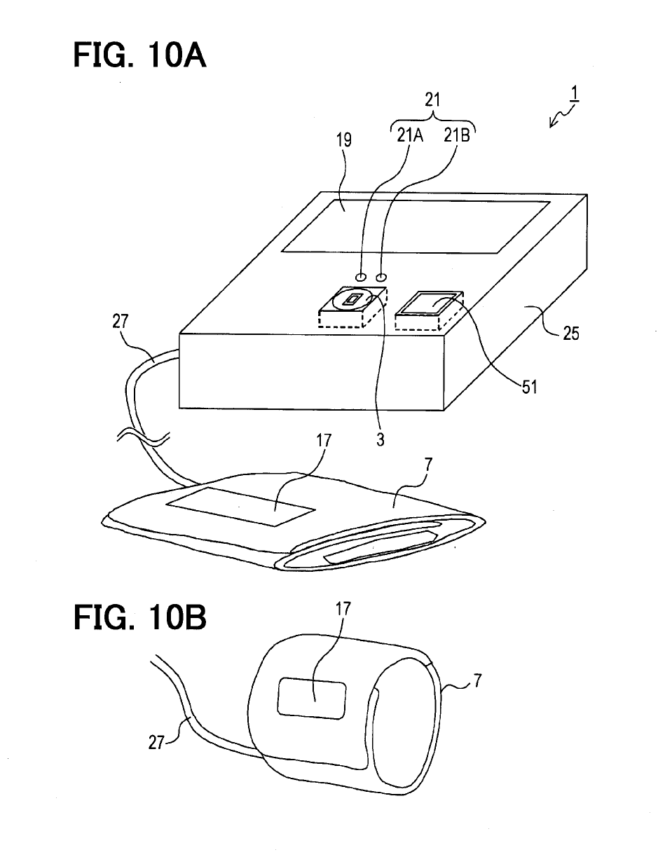

The sphygmomanometer 1 in the present embodiment has, as shown in FIG. 10, a difference Δ compu-mem switch 51 separately from the measurement switch 3. This difference Δ compu-mem switch 51 is a finger-press switch which is pressed by the patient with his/her finger, for the computation and memorization operation of the difference Δ.

2. Process Performed by Sphygmomanometer 1

The process performed by the sphygmomanometer 1 is the same as the one in the first embodiment fundamentally. However, in Step 17, it is determined whether an operation is performed on the difference Δ compu-mem switch 51 within a predetermined time from the display in Step 16. When the operation is performed, the process proceeds to Step 18, and, when the operation is not performed, this process is ended.

In other words, in the present embodiment, the operation on the difference Δ compu-mem switch 51 has the same effect as the second step operation in the first embodiment.

3. Effects of Sphygmomanometer 1

(1) The sphygmomanometer 1 of the present embodiment exerts substantially the same effect as the first embodiment.

(2) The sphygmomanometer 1 of the present embodiment has a simpler configuration of the measurement switch 3 than the one in the first embodiment.

### Other Embodiments

(1) In the first and second embodiments, a slide type switch, a pressure-sensitive switch, a switch on a touch panel, etc. may be used to replace the measurement switch 3.

(2) In the first and second embodiments, the sensor of other types may be used in place of the cuff wear sensor 17. For example, the cuff wear sensor may detect the wearing of the cuff based on the temperature (i.e., a patient's body temperature), or may detect the wearing of the cuff based on the pressure on the inner circumference surface of the cuff.

(3) All or a part of the configuration in the first and second embodiments may be combined.

(4) The measurement switch 3 may be operated by a part of the patient body other than the finger. For example, a palm, a leg, a back of the hand, a knee may be used to operate the switch 3. In such case, the part used to operate the switch 3 is also used to detect the pulse wave signal by using the pulse wave sensor 5.

Although the present disclosure has been fully described in connection with preferred embodiment thereof with reference to the accompanying drawings, it is to be noted that various changes and modifications will become apparent to those skilled in the art, and such changes, modifications, and summarized scheme are to be understood as being within the scope of the present disclosure as defined by appended claims.

What is claimed is:

1. A sphygmomanometer comprising:
an operation part that is operated by a part of a patient body;
a pulse wave sensor detecting a pulse wave signal indicative of a pulse wave in the part of the patient body that is operating the operation part;
a first manometer measuring a blood pressure based on the pulse wave signal detected by the pulse wave sensor;
a second manometer measuring the blood pressure with a cuff that is worn by the patient;
a cuff wearing detector detecting a wearing of the cuff by the patient; and
a controller (i) measuring the blood pressure with the first manometer when the operation part is being operated and a duration of the detection of the pulse wave signal by the pulse wave sensor is equal to or greater than a preset time, and (ii) measuring the blood pressure with the second manometer when the operation part is being operated and the cuff wearing detector detects the wearing of the cuff.

2. The sphygmomanometer of claim 1, further comprising:
a difference calculator calculating and memorizing a difference between a measurement value of the first manometer and a measurement value of the second manometer; and
a correction part correcting the measurement value according to the difference.

3. The sphygmomanometer of claim 2, wherein
the operation part is operated by a first step and a second step, and a direction to operate the first step and a direction to operate the second step is a same direction,
when the first step is operated, the controller performs a measurement by using one or both of the first manometer and the second manometer, and
when the second step is operated, the difference calculator performs a calculation and memorizes the difference.

4. The sphygmomanometer of claim 1, wherein
the cuff wearing detector detects the wearing of the cuff based on a circumferential stretch amount of the cuff.

5. The sphygmomanometer of claim 1, wherein
the part of the patient body is a part of an arm that is opposite to a cuff-wearing arm.

6. A sphygmomanometer comprising:
an operation part that is operated by a part of a patient body;
a pulse wave sensor detecting a pulse wave signal indicative of a pulse wave in the part of the patient body that is operating the operation part;
a first manometer measuring a blood pressure based on the pulse wave signal detected by the pulse wave sensor;
a second manometer measuring the blood pressure with a cuff that is worn by the patient;
a cuff wearing detector detecting a wearing of the cuff by the patient; and
a controller (i) measuring the blood pressure with the first manometer when the operation part is being operated and a duration of the detection of the pulse wave signal by the pulse wave sensor is equal to or greater than a preset time, (ii) measuring the blood pressure with the second manometer when the operation part is being operated, a duration of the detection of the pulse wave signal by the pulse wave sensor is shorter than the preset time, and the cuff wearing detector detects the wearing of the cuff, or (iii) measuring the blood pressure with the first manometer and the second manometer when the operation part is being operated, a duration of the detection of the pulse wave signal by the pulse wave sensor is equal to or greater than a preset time, and the cuff wearing detector detects the wearing of the cuff.

7. The sphygmomanometer of claim 6, further comprising:
a difference calculator calculating and memorizing a difference between a measurement value of the first manometer and a measurement value of the second manometer; and
a correction part correcting the measurement value according to the difference.

8. The sphygmomanometer of claim 7, wherein
the operation part operated by a first step and a second step, and a direction to operate the first step and a direction to operate the second step is a same direction,
when the first step is operated, the controller performs a measurement by using one or both of the first manometer and the second manometer, and
when the second step is operated, the difference calculator performs a calculation and memorizes the difference.

9. The sphygmomanometer of claim 6, wherein
the cuff wearing detector detects the wearing of the cuff based on a circumferential stretch amount of the cuff.

10. The sphygmomanometer of claim 6, wherein
the part of the patient body is a part of an arm that is opposite to a cuff-wearing arm.
